(19) United States
(12) Patent Application Publication
(10) Pub. No.: US 2018/0110405 A1
(43) Pub. Date: Apr. 26, 2018

(54) IMAGE PICKUP APPARATUS FOR ENDOSCOPE

(71) Applicant: OLYMPUS CORPORATION, Tokyo (JP)

(72) Inventor: Kazuhiro Yoshida, Okaya-shi (JP)

(73) Assignee: OLYMPUS CORPORATION, Tokyo (JP)

(21) Appl. No.: 15/848,991

(22) Filed: Dec. 20, 2017

Related U.S. Application Data
(63) Continuation of application No. PCT/JP2015/067849, filed on Jun. 22, 2015.

(51) Int. Cl. A61B 1/05; A61B 1/00

(52) U.S. Cl. CPC A61B 1/05; A61B 1/0661; A61B 1/00009; A61B 1/00096

(57) ABSTRACT

An image pickup apparatus for endoscope includes: an image pickup optical system; a right angle prism configured to receive light from the image pickup optical system; and an image pickup substrate rectangular in plan view with a thickness equal to or greater than 20 μm and equal to or smaller than 100 μm, in which the right angle prism is adhered to a first main surface 60SA, and a light receiving portion is formed below the right angle prism, wherein a groove is formed on a second main surface of the image pickup substrate, and a direction of the groove is inclined more than 45 degrees relative to a short axis direction.

### CROSS REFERENCE TO RELATED APPLICATION

This application is a continuation application of PCT/JP2015/067849 filed on Jun. 22, 2015, the entire contents of which are incorporated herein by this reference.

### BACKGROUND OF INVENTION

### 1. Field of the Invention

The present invention relates to an image pickup apparatus for endoscope including an image pickup optical system, an optical path conversion element configured to receive light from the image pickup optical system, and an image pickup substrate in which the optical path conversion element is adhered to a first main surface.

### 2. Description of the Related Art

An electronic endoscope provided with an image pickup apparatus including a solid-state image pickup device, such as a CMOS light receiving element, on a distal end portion of an insertion portion is widely used. In an endoscope for medical use, a flexible elongated insertion portion including an image pickup apparatus on a distal end portion is inserted into a body cavity of a subject, such as a patient, to perform observation and the like of a site to be examined.

An image pickup apparatus for endoscope is disclosed in U.S. Pat. No. 8,913,112 (Japanese Patent No. 5080695), wherein a prism configured to receive light from an image pickup optical system is adhered to a light receiving surface of an image pickup substrate.

Reduction in a diameter of the insertion portion is demanded to reduce invasiveness by the endoscope. To reduce the diameter, it is effective to thinly process the image pickup substrate.

### SUMMARY OF THE INVENTION

An embodiment of the present invention provides an image pickup apparatus for endoscope including: an image pickup optical system; an optical path conversion element configured to receive light from the image pickup optical system and bend an optical path; and an image pickup substrate rectangular in plan view with a thickness equal to or greater than 20 μm and equal to or smaller than 100 μm, in which the optical path conversion element is adhered to a first main surface, and a light receiving portion configured to receive the light bent by the optical path conversion element is formed, wherein at least one groove is formed on a second main surface of the image pickup substrate, and a direction of the groove is inclined more than 45 degrees relative to a short axis direction of the image pickup substrate.

Another embodiment provides a manufacturing method of an image pickup apparatus for endoscope, the image pickup apparatus for endoscope including: an image pickup optical system; an optical path conversion element configured to receive light from the image pickup optical system and bend an optical path; and an image pickup substrate rectangular in plan view with a thickness equal to or greater than 20 μm and equal to or smaller than 100 in which the optical path conversion element is adhered to a first main surface, and a light receiving portion configured to receive the light bent by the optical path conversion element is formed, the manufacturing method including: forming a plurality of light receiving portions on a first main surface of a semiconductor substrate; manufacturing a plurality of image pickup substrates by cutting the semiconductor substrate; disposing the plurality of image pickup substrates on a grinding machine such that directions of saw marks to be formed are same; and forming grooves inclined more than 45 degrees relative to a short axis direction of the plurality of image pickup substrates by grinding second main surfaces of the plurality of image pickup substrates.

### BRIEF DESCRIPTION OF THE DRAWINGS

FIG. 1 is an external view of an endoscope system including an image pickup apparatus for endoscope according to an embodiment;

FIG. 2A is a cross-sectional view of a distal end portion of an insertion portion of the image pickup apparatus for endoscope in a direction parallel to a major axis direction according to the embodiment;

FIG. 2B is a cross-sectional view of the distal end portion of the insertion portion of the image pickup apparatus for endoscope in a direction perpendicular to the major axis direction according to the embodiment;

FIG. 3 is a cross-sectional view of an image pickup substrate of the image pickup apparatus for endoscope according to a first embodiment;

FIG. 4 is a perspective view of the image pickup substrate of the image pickup apparatus for endoscope according to the first embodiment;

FIG. 5A is a side view of the image pickup substrate of the image pickup apparatus for endoscope according to the first embodiment;

FIG. 5B is a diagram showing a second main surface of the image pickup substrate of the image pickup apparatus for endoscope according to the first embodiment;

FIG. 6A is a side view of an image pickup substrate of an image pickup apparatus for endoscope according to modification 1 of the first embodiment;

FIG. 6B is a diagram showing a second main surface of the image pickup substrate of the image pickup apparatus for endoscope according to modification 1 of the first embodiment;

FIG. 7A is a side view of an image pickup substrate of an image pickup apparatus for endoscope according to modification 2 of the first embodiment;

FIG. 7B is a diagram showing a second main surface of the image pickup substrate of the image pickup apparatus for endoscope according to modification 2 of the first embodiment;

FIG. 8 is a flowchart for describing a manufacturing method of an image pickup apparatus for endoscope according to a second embodiment;

FIG. 9 is a schematic view for describing an infeed-type grinding machine;

FIG. 10 is a diagram showing directions of saw marks formed by the infeed-type grinding machine;

FIG. 11 is a diagram for describing disposition of grinding workpieces in the manufacturing method of the image pickup apparatus for endoscope according to the second embodiment;

FIG. 12 is a diagram for describing directions of saw marks of grinding workpieces in the manufacturing method of the image pickup apparatus for endoscope according to the second embodiment;

FIG. 13A is a side view of an image pickup substrate of the image pickup apparatus for endoscope according to the second embodiment;

FIG. 13B is a diagram showing directions of saw marks on a second main surface of the image pickup substrate according to the second embodiment;

FIG. 14 is a top view of the grinding workpieces of the image pickup apparatus for endoscope according to the second embodiment;

FIG. 15 is a diagram for describing directions of saw marks in a manufacturing method of an image pickup apparatus for endoscope according to a modification of the second embodiment; and

FIG. 16 is a diagram for describing disposition of grinding workpieces in the manufacturing method of the image pickup apparatus for endoscope according to the modification of the second embodiment.

### DETAILED DESCRIPTION OF THE PREFERRED EMBODIMENTS

### First Embodiment

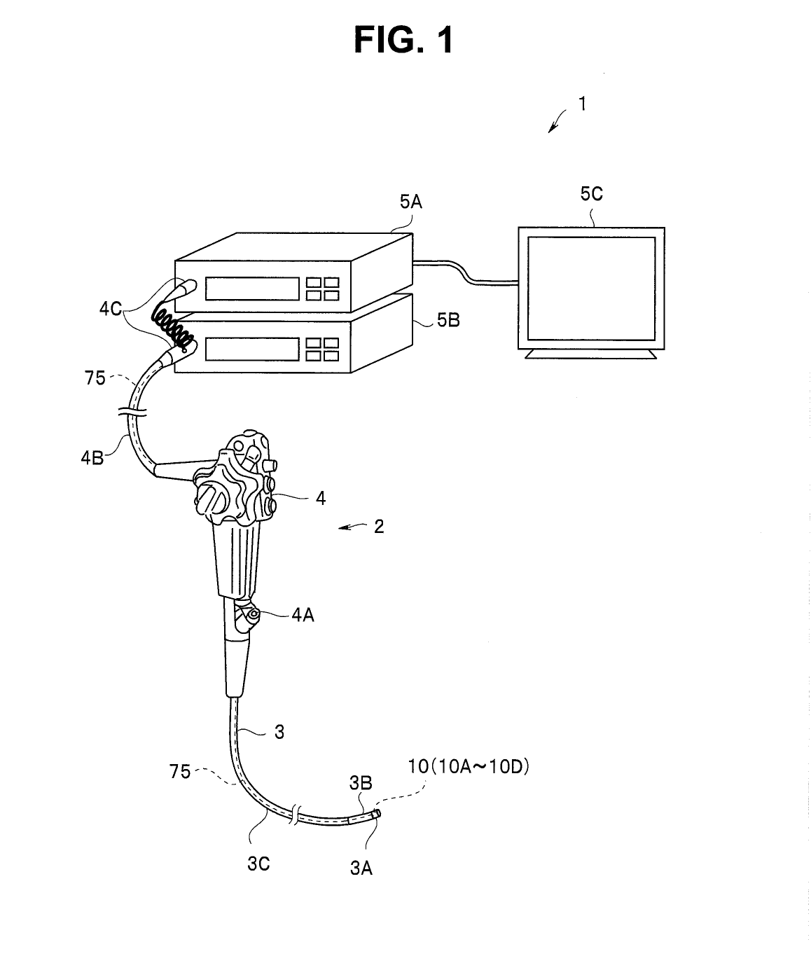

An endoscope system 1 including an endoscope 2 provided with an image pickup apparatus for endoscope (hereinafter, also referred to as “image pickup apparatus”) 10 according to a first embodiment of the present invention will be described with reference to FIG. 1.

Note that the drawings are schematic drawings, and a relationship between thickness and width of each part, a ratio of the thickness of respective parts, and the like are different from the reality. The relationship or the ratio of dimensions may be different between the drawings in some parts of the drawings.

As shown in FIG. 1, the endoscope system 1 includes the endoscope 2, a processor 5A, a light source apparatus 5B, and a monitor 5C. An elongated insertion portion 3 is inserted into a body cavity of a subject, and the endoscope 2 picks up an in-vivo image of the subject and outputs an image pickup signal.

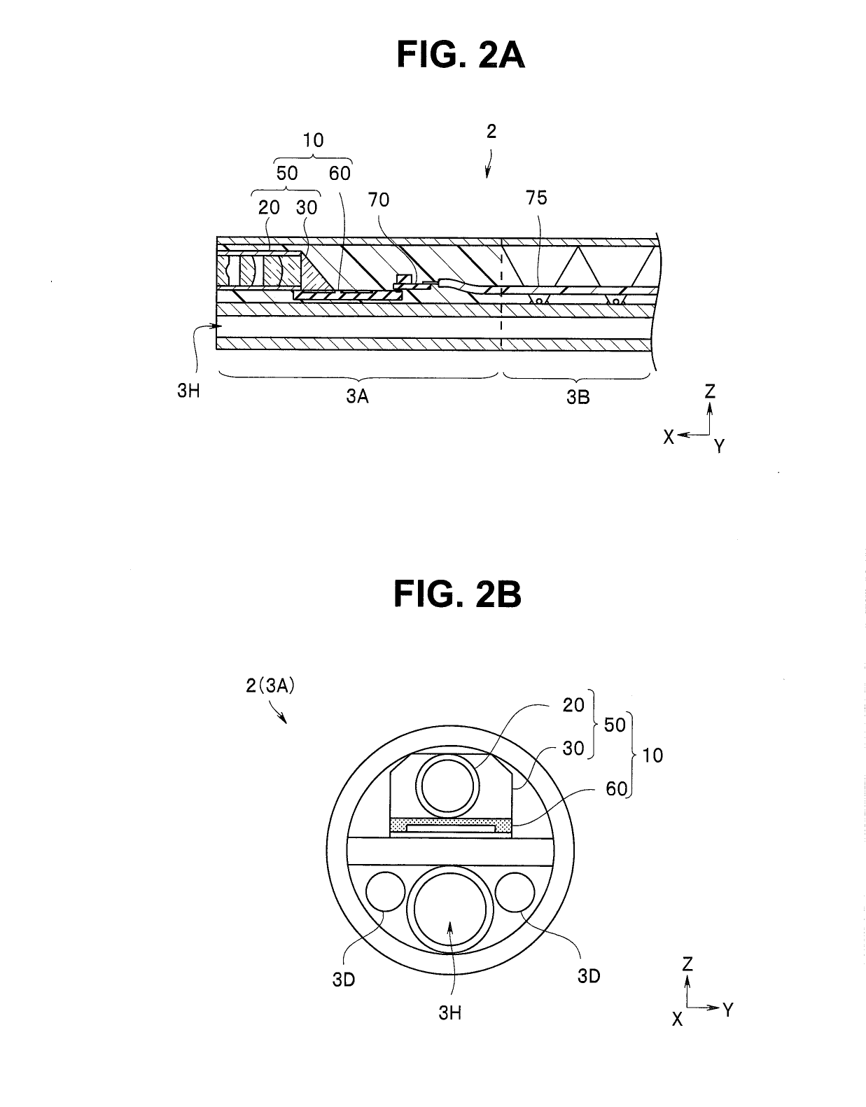

An operation portion 4 provided with various buttons for operating the endoscope 2 is provided on a proximal end side of the insertion portion 3 of the endoscope 2. The operation portion 4 includes a treatment instrument insertion port 4A of a channel 3H (see FIG. 2) for inserting a treatment instrument, such as biological forceps, an electric knife, and an inspection probe, into a body cavity of the subject.

The insertion portion 3 is configured by a distal end portion 3A provided with the image pickup apparatus 10, a bendable bending portion 3B continuously provided on a proximal end side of the distal end portion 3A, and a flexible tube portion 3C continuously provided on a proximal end side of the bending portion 3B. The bending portion 3B is bent by operation of the operation portion 4.

A signal cable 75 connected to the image pickup apparatus 10 of the distal end portion 3A is inserted into a universal cord 4B arranged on a proximal end portion side of the operation portion 4.

The universal cord 4B is connected to the processor 5A and the light source apparatus 5B through a connector 4C. The processor 5A controls the entire endoscope system 1 and applies signal processing to an image pickup signal outputted by the image pickup apparatus 10 to output an image signal. The monitor 5C displays the image signal outputted by the processor 5A.

The light source apparatus 5B includes, for example, a white LED. White light emitted by the light source apparatus 5B is guided to the distal end portion 3A through a light guide (not shown) inserted into the universal cord 4B and the insertion portion 3, and the white light illuminates an object.

Next, a configuration of the distal end portion 3A of the endoscope 2 will be described with reference to FIGS. 2A and 2B.

The image pickup apparatus 10, the treatment instrument channel 3H, and the like are arranged on the distal end portion 3A. An illumination optical system 3D configured to emit illumination light is also arranged on the distal end portion 3A.

The image pickup apparatus 10 includes an optical unit 50 and an image pickup substrate 60, and the optical unit 50 includes an image pickup optical system 20 and a prism 30 that is an optical path conversion element. A back end portion of the image pickup apparatus 10 is sealed by a sealing resin 72.

The image pickup substrate 60 provided with the surface-mounted optical unit 50 is connected to the signal cable 75 through a wiring board 70. Note that an outer circumference of the distal end portion 3A is covered by a soft cover tube not shown.

The distal end portion 3A of the endoscope 2 has a small diameter equal to or smaller than 8 mm, for example. Note that the endoscope of the embodiment may have a smaller diameter without the arrangement of the treatment instrument channel 3H and may be dedicated to observation.

### <Configuration of Image Pickup Apparatus>

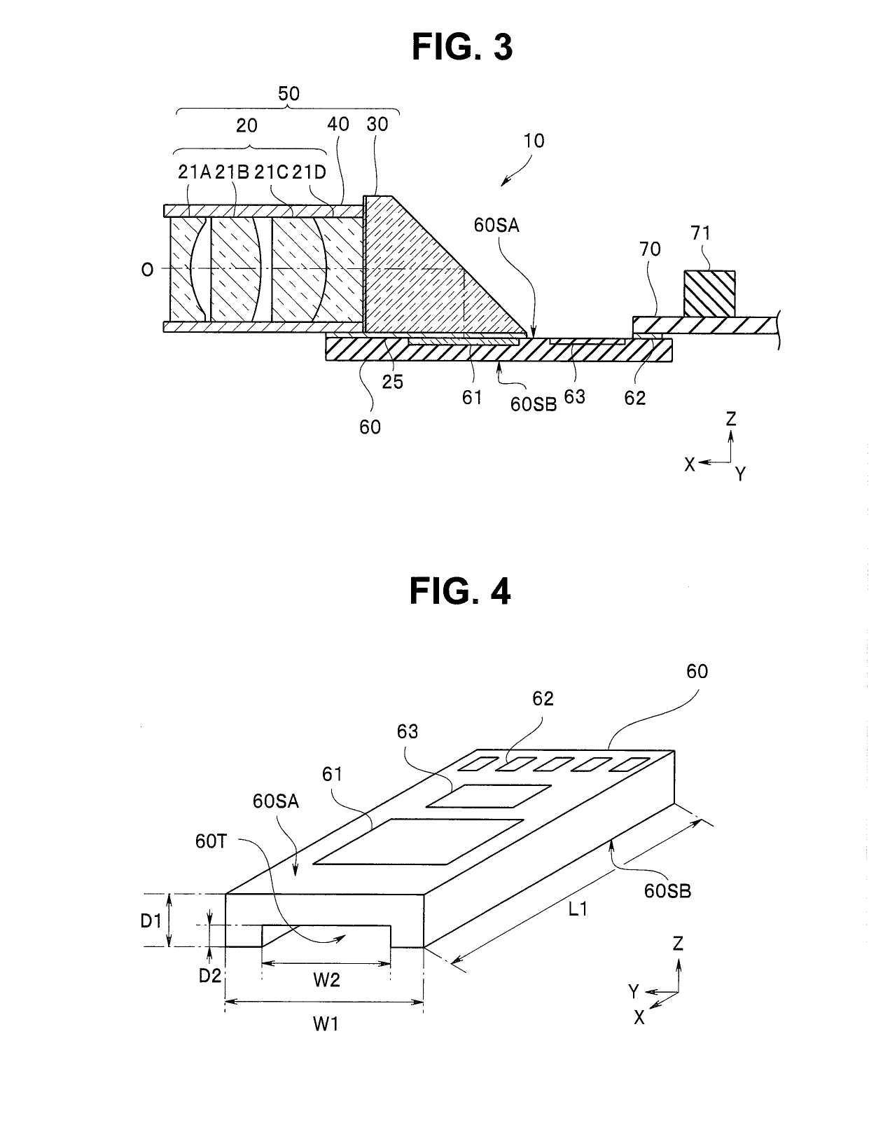

Next, a configuration of the image pickup apparatus 10 of the present embodiment will be described in detail with reference to FIGS. 3 and 4.

As shown in FIGS. 3 and 4, the image pickup apparatus 10 is a so-called “horizontal type” in which an optical axis O of the image pickup optical system 20 is parallel to a first main surface 60SA of the image pickup substrate 60.

The optical unit 50 includes: a plurality of lenses 21A to 21D fixed by a lens frame 40; and the prism 30.

The image pickup substrate 60 rectangular in plan view including the first main surface 60SA and a second main surface 60SB is constituted by a semiconductor made of silicon or the like in which a light receiving portion 61 and a signal processing circuit 63 are formed on the first main surface 60SA. The light receiving portion 61 is a CMOS (complementary metal oxide semiconductor) type semiconductor circuit or a CCD (charge coupled device). A plurality of electrode pads 62 electrically connected to the light receiving portion 61 are arranged on an end portion of the image pickup substrate 60. The wiring board 70 provided with an electronic component 71 is bonded to the electrode pads 62. A plurality of connection electrodes (not shown) of a back end portion of the wiring board 70 are bonded to the signal cable 75. Solder bonding or ultrasonic bonding is used for the bonding.

The image pickup optical system 20 and the prism 30 of the image pickup apparatus 10 are adhered to the first main surface 60SA of the image pickup substrate 60 through an adhesive layer 25 made of an ultraviolet curable resin. An ultraviolet curable transparent resin is also filled between the image pickup optical system 20 and the prism 30.

The light entering the optical unit 50 is condensed by the image pickup optical system 20, and the light enters the prism 30 that is an optical path conversion element. The prism 30 reflects the incident light parallel to the first main surface 60SA from the image pickup optical system 20 to convert an optical path of the incident light by 90 degrees in a direction perpendicular to the first main surface 60SA and emits the light to the light receiving portion 61. That is, the prism 30 as an optical path conversion element has an optical effect of bending the optical path of the light emitted from the image pickup optical system 20 by 90 degrees to cause the light to enter the light receiving portion 61. In other words, the prism 30 receives the light from the image pickup optical system 20 and bends the optical path. Note that the optical path conversion element is not limited to the right angle prism 30, and the optical path conversion element may be a mirror (reflective surface).

The light receiving portion 61 receives the light reflected by the prism 30 and converts the received light into an image pickup signal. The image pickup signal outputted by the image pickup apparatus 10 is transmitted to the processor 5A through the wiring board 70 and the signal cable 75.

As shown in FIG. 4, the image pickup substrate 60 of the image pickup apparatus 10 is processed to reduce the diameter of the insertion portion 3 such that a thickness D1 of the image pickup substrate 60 is equal to or greater than 20 μm and equal to or smaller than 100 μm, or about 50 μm for example. On the other hand, a length L1 in a major axis direction of the image pickup substrate 60 rectangular in plan view is, for example, about 3000 μm, and a width W1 in a short axis direction is about 1000 μm. Therefore, when stress is applied to the image pickup substrate 60 during manufacturing, particularly, during insertion into the distal end portion 3A, a crack or the like may occur on a side surface in the major axis direction, and a manufacturing yield of the image pickup apparatus 10 may be reduced.

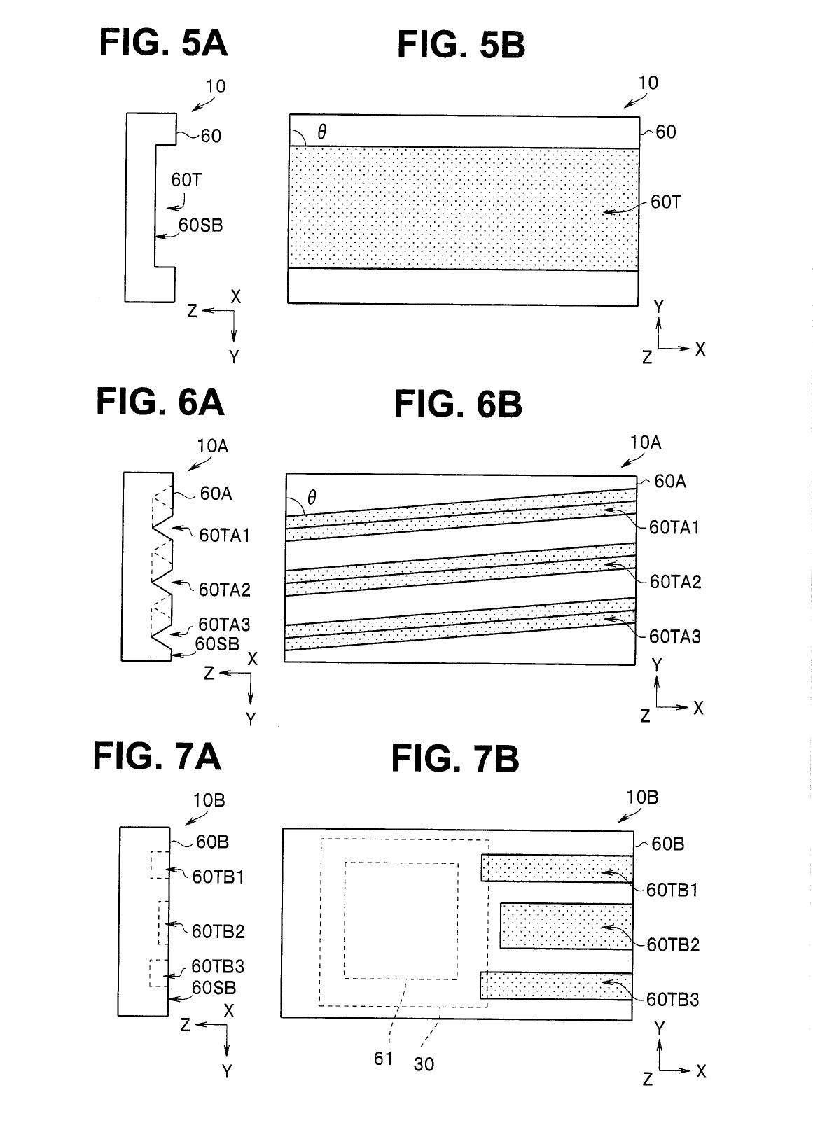

As shown in FIGS. 4, 5A, and 5B, a groove 60T is formed on the second main surface (bottom surface/back surface) 60SB in the image pickup substrate 60, and the direction of the groove 60T is orthogonal to the short axis direction (Y direction). In other words, the groove 60T is parallel to the major axis direction (X direction) of the image pickup substrate 60 rectangular in plan view and is inclined 90 degrees relative to the short axis direction (Y direction).

A depth D2 of the groove 60T is 30% of the thickness D1 of the image pickup substrate 60, and a width W2 is 70% of the width W1 of the image pickup substrate 60.

Note that if the depth D2 of the groove 60T is equal to or greater than 10% of the thickness D1 of the image pickup substrate 60, the advantageous effect is prominent. If the depth D2 is equal to or smaller than 50% of the thickness D1, the light receiving portion 61 and the like formed on the first main surface side are not adversely affected. If equal to or greater than 5% of the width W1 of the image pickup substrate 60 remains on both sides of the width W2 of the groove 60T, the strength is secured. If the width W2 is equal to or greater than 50% of the width W1, the advantageous effect is prominent. Therefore, it is preferable that the width W2 of the groove 60T be equal to or greater than 50% and equal to or smaller than 90% of the width W1.

Although the groove 60T can be formed by mechanical processing such as grinding, it is preferable to form the groove 60T by an etching process through a resist mask in a wafer state including a plurality of image pickup substrates. The etching may be dry etching or wet etching.

Although the thickness D1 of the image pickup substrate 60 is thin, the groove 60T is provided. Therefore, even if stress is applied through the wiring board 70, a crack or the like does not occur on the side surface, and the manufacturing yield of the image pickup apparatus 10 is high.

### <Modifications of First Embodiment>

Next, image pickup apparatuses for endoscope 10A and 10B according to modifications 1 and 2 of the first embodiment will be described. The image pickup apparatuses for endoscope 10A and 10B are similar to the image pickup apparatus for endoscope 10 and have the same functions. Therefore, the same reference signs are provided to the same components, and the description will not be repeated.

### <Modification 1>

As shown in FIGS. 6A and 6B, three grooves 60TA (60TA1, 60TA2, and 60TA3) are formed on the second main surface 60SB of an image pickup substrate 60A of the image pickup apparatus 10A.

An inclination angle θ of the grooves 60TA with respect to the short axis direction (Y direction) of the image pickup substrate 60A is an inclination of about 65 degrees, and the grooves 60TA do not have openings on the major axis side surface. Furthermore, the grooves 60TA are V-grooves with a triangular cross section.

Although the thickness of the image pickup substrate 60A is thin just like the image pickup substrate 60, the grooves 60TA are provided. Therefore, even if stress is applied, a crack or the like does not occur on the side surface, and the manufacturing yield of the image pickup apparatus 10A is high.

That is, a plurality of grooves may be formed on the image pickup substrate. The grooves are not limited to the V-grooves with a triangular cross-sectional shape, and the shape may be rectangular, semicircular, trapezoidal, or the like. The direction of the grooves has an advantageous effect of preventing occurrence of a crack on the side surface as long as no opening on the major axis side surface exists and the inclination angle θ is greater than 45 degrees (less than 135 degrees). The inclination angle θ of the grooves is preferably equal to or greater than 60 degrees (equal to or smaller than 120 degrees), more preferably equal to or greater than 80 degrees (equal to or smaller than 100 degrees), and most preferably 90 degrees. Note than the grooves may be curved, and the inclination angles θ of the plurality of grooves may be different.

### <Modification 2>

As shown in FIGS. 7A and 7B, three grooves 60TB (60TB1, 60TB2, and 60TB3) are formed on the second main surface 60SB of an image pickup substrate 60B in the image pickup apparatus 10B. The grooves 60TB are not formed in a region facing the light receiving portion 61 where the image pickup optical system 20 (prism 30) is adhered, that is, a region equivalent to a back surface of the light receiving portion 61. The groove 60TB2 has a shallower depth, a wider width, and a shorter length than the groove 60TB1. That is, the shapes and the like of the plurality of grooves may not be the same.

In the image pickup apparatus with the image pickup optical system 20 adhered to the first main surface, the image pickup optical system 20 has a function of reinforcing the mechanical strength of the image pickup substrate 60B. Therefore, even if the image pickup substrate 60B has a part where the grooves 60TB are not formed, a crack is unlikely to occur during, for example, the insertion into the distal end portion 3A. Furthermore, the grooves are not formed in the region facing the light receiving portion 61, and the light receiving portion 61 is not adversely affected.

### Second Embodiment

Next, an image pickup apparatus for endoscope 10C according to a second embodiment will be described. The image pickup apparatus 10C is similar to the image pickup apparatus 10 of the first embodiment. Therefore, the same reference signs are provided to the components with the same functions, and the description will not be repeated.

An image pickup substrate 60C (see FIGS. 13A and 13B) of the image pickup apparatus 10C is rectangular in plan view with a thickness equal to or greater than 20 μm and equal to or smaller than 100 μm just like the image pickup substrate 60. A plurality of grooves 60TC with a maximum inclination angle θ of more than 45 degrees (less than 135 degrees) relative to the short axis direction are formed on the second main surface of the image pickup substrate 60C. Here, the grooves 60TC are saw marks formed by grinding for thinning the image pickup substrate.

Although the thickness of the image pickup substrate 60C is thin in the image pickup apparatus 10C, the plurality of grooves 60TC are provided. Therefore, even if stress is applied, a crack or the like does not occur on the side surface, and the manufacturing yield is high.

Note that an advantageous effect of preventing occurrence of a crack on the side surface exists as long as the inclination angle θ of the plurality of grooves 60TC is more than 45 degrees (less than 135 degrees). It is more preferable that the inclination angle θ be equal to or greater than 60 degrees (equal to or smaller than 120 degrees).

### <Manufacturing Method of Image Pickup Apparatus>

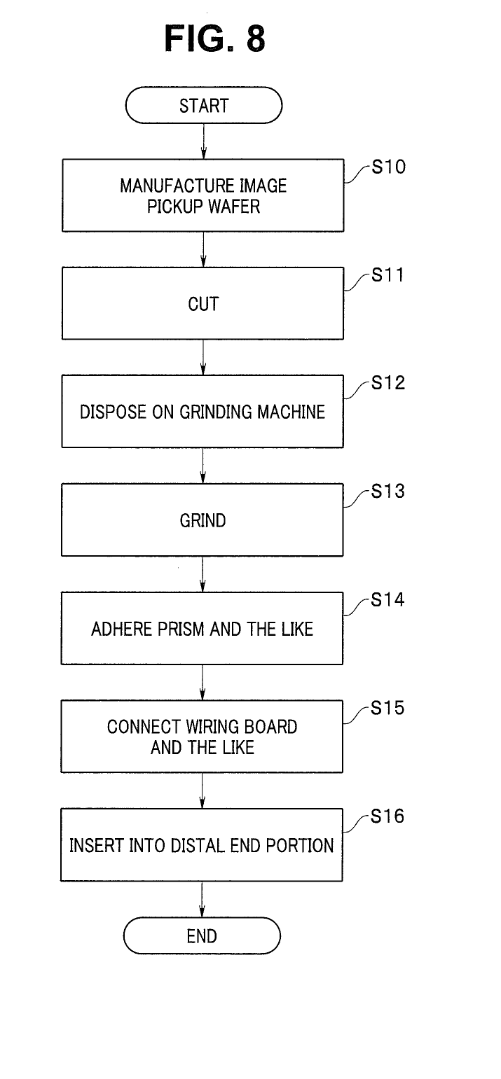

Next, a manufacturing method of the image pickup apparatus 10C will be simply described along with a flowchart of FIG. 8.

### <Step S10>

The plurality of light receiving portions 61, the plurality of signal processing circuits 63, and the like are formed on a silicon wafer by using a well-known semiconductor process. Note that the thickness of the silicon wafer of 300 mmϕ is, for example, 775 μm.

### <Step S11>

The silicon wafer is cut to manufacture a plurality of image pickup substrates 60C1, each provided with the light receiving portion 61, the signal processing circuit 63, and the like.

### <Step S12>

The image pickup substrates 60C1 are thick with a thickness of 775 μm. The image pickup substrates 60C1 need to be processed such that the thickness D1 is equal to or greater than 20 μm and equal to or smaller than 100 μm in order to reduce the diameter of the insertion portion 3.

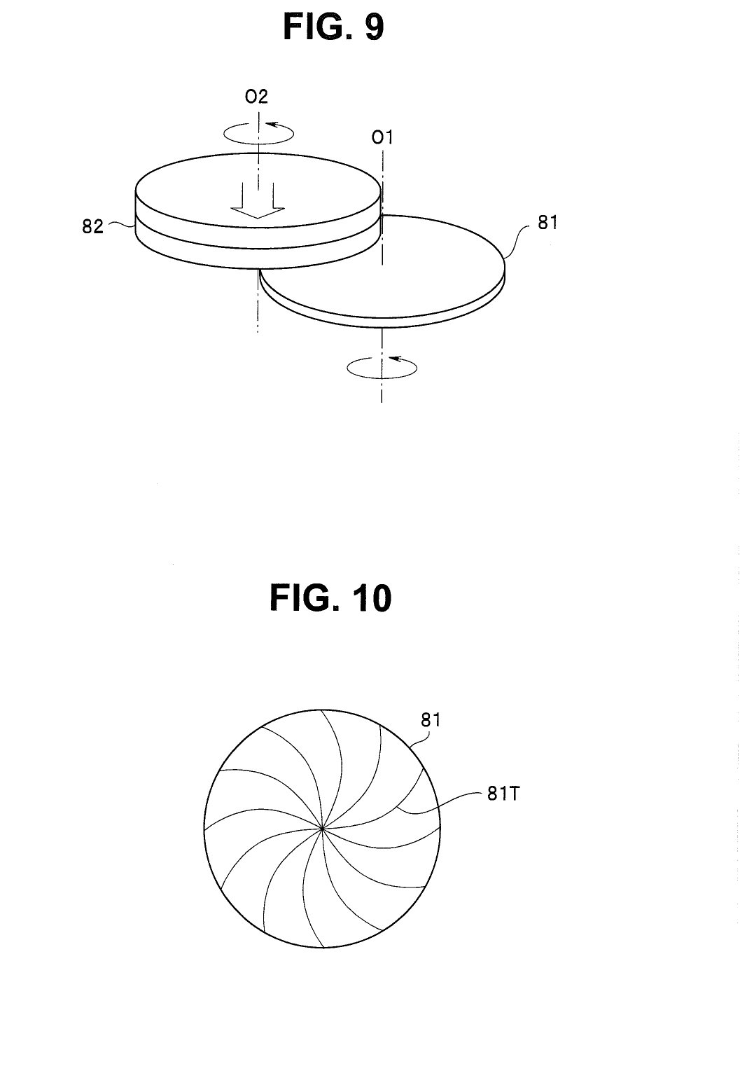

To thin the image pickup substrates 60C1, grinding is preferable in terms of manufacturing efficiency. FIG. 9 shows an example of a grinding machine 80. The infeed-type grinding machine 80 includes: a holding plate 81 provided with the image pickup substrates 60C1 that are workpieces; and a grinder 82 configured to grind the image pickup substrates 60C1 disposed on the holding plate 81. The grinder 82 is provided with, for example, a plurality of grindstones containing diamond abrasive grains. The image pickup substrates 60C1 are fixed to the holding plate 81 by a protective tape or the like. The grinding machine 80 is a centerless type in which a rotation axis O1 of the holding plate 81 and a rotation axis O2 of the grinder 82 do not coincide.

In the grinding machine 80, radial saw marks (grooves 81T) are formed on the workpieces of the holding plate 81 as shown in FIG. 10.

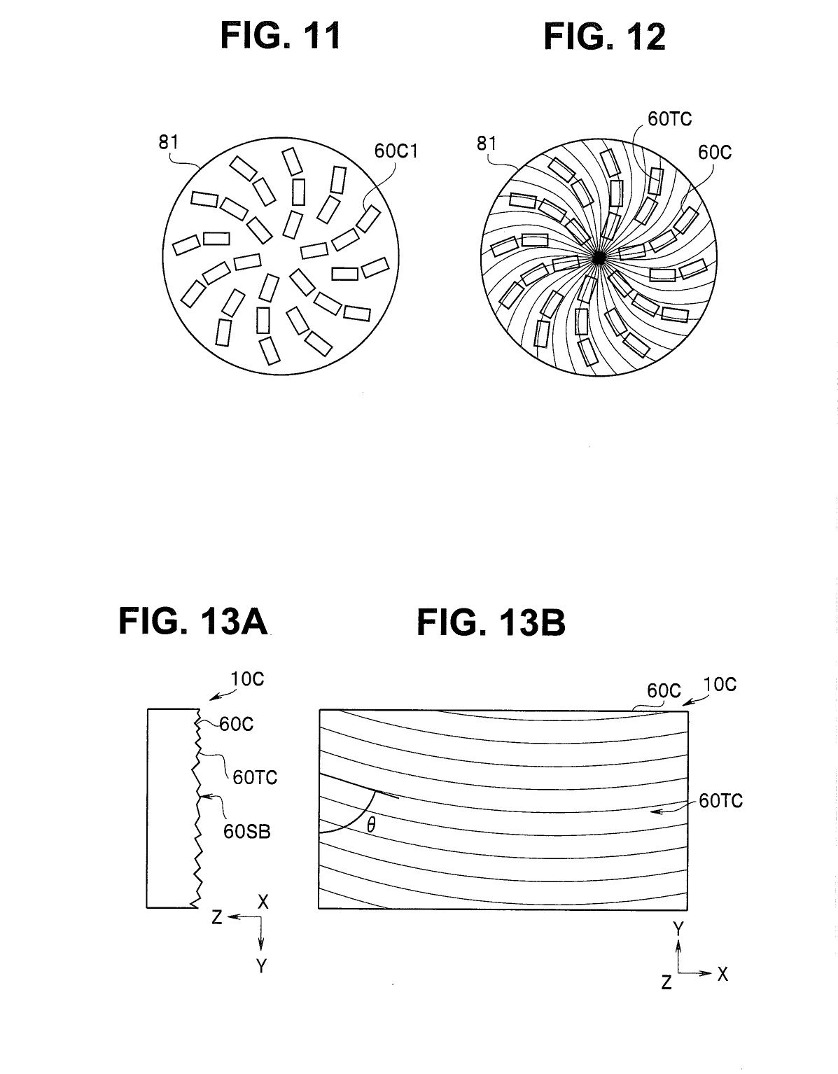

In the manufacturing method of the image pickup apparatus 10C of the present embodiment, the plurality of image pickup substrates 60C1 are disposed on the holding plate 81 such that the major axis direction is parallel to the direction of the formation of the saw marks as shown in FIG. 11. That is, in a manufacturing method of a conventional image pickup apparatus, grinding is performed in the state of semiconductor substrate (silicon wafer) including a plurality of image pickup substrates. On the other hand, the silicon wafer is cut, and the image pickup substrates 60C1 are rearranged in the manufacturing method of the image pickup apparatus 10C of the embodiment.

### <Step S13>

The image pickup substrates 60C1 are ground such that the thickness is equal to or greater than 20 μm and equal to or smaller than 100 μm, and a surface roughness (JIS B 060: ten-point average roughness, measurement length 1 mm) Rz in the direction orthogonal to the direction of the saw marks (grooves) of the second main surface 60SB is equal to or greater than 1 μm and equal to or smaller than 5 μm. It is more preferable that the surface roughness Rz be equal to or smaller than 2 μm.

If the surface roughness is equal to or greater than the range, the advantageous effect of preventing occurrence of a crack or the like is prominent. If the surface roughness is equal to or smaller than the range, problems caused by the saw marks do not occur.

As shown in FIGS. 12, 13A, and 13B, the plurality of grooves 60TC that are similarly inclined more than 45 degrees relative to the short axis direction are formed on the plurality of ground image pickup substrates 60C.

If the surface roughness is within the range, and the direction of the plurality of grooves 60TC is greater than 45 degrees (less than 135 degrees), an advantageous effect of preventing occurrence of a crack on the side surface exists. The inclination angle θ of the grooves 60TC is preferably equal to or greater than 60 degrees (equal to or smaller than 120 degrees) and is more preferably equal to or greater than 80 degrees (equal to or smaller than 100 degrees). Note that although the grooves 60TC are curves, the inclination angle θ is within the range in all of the ranges of the grooves 60TC.

### <Step S14>

The image pickup optical system 20, the prism 30, and the like are manufactured according to specifications. For example, the lens 21 and the prism 30 are made of glass or a transparent resin, and the lens frame 40 is made of metal.

The prism 30 and the like held by a suction tool are then aligned with the light receiving portion 61 applied with an adhesive made of an ultraviolet curable transparent resin. The adhesive is cured when ultraviolet rays are applied, and the prism 30 is adhered to the image pickup substrate 60 through the adhesive layer 25.

### <Step S15>

The wiring board 70 is connected to the image pickup substrate 60C. For example, the electrode pads 62 are bonded by soldering to electrodes of the wiring board 70. The signal cable 75 is further bonded to the wiring board 70. The wiring board 70, to which the signal cable 75 is bonded, may be bonded to the image pickup substrate 60C.

### <Step S16>

The image pickup substrate 60C to which the prism 30 and the like are adhered is inserted into the distal end portion 3A. In this case, even if stress is applied to the image pickup substrate 60C, a crack or the like does not occur on the side surface in the major axis direction, and the manufacturing yield of the image pickup apparatus 10C is high.

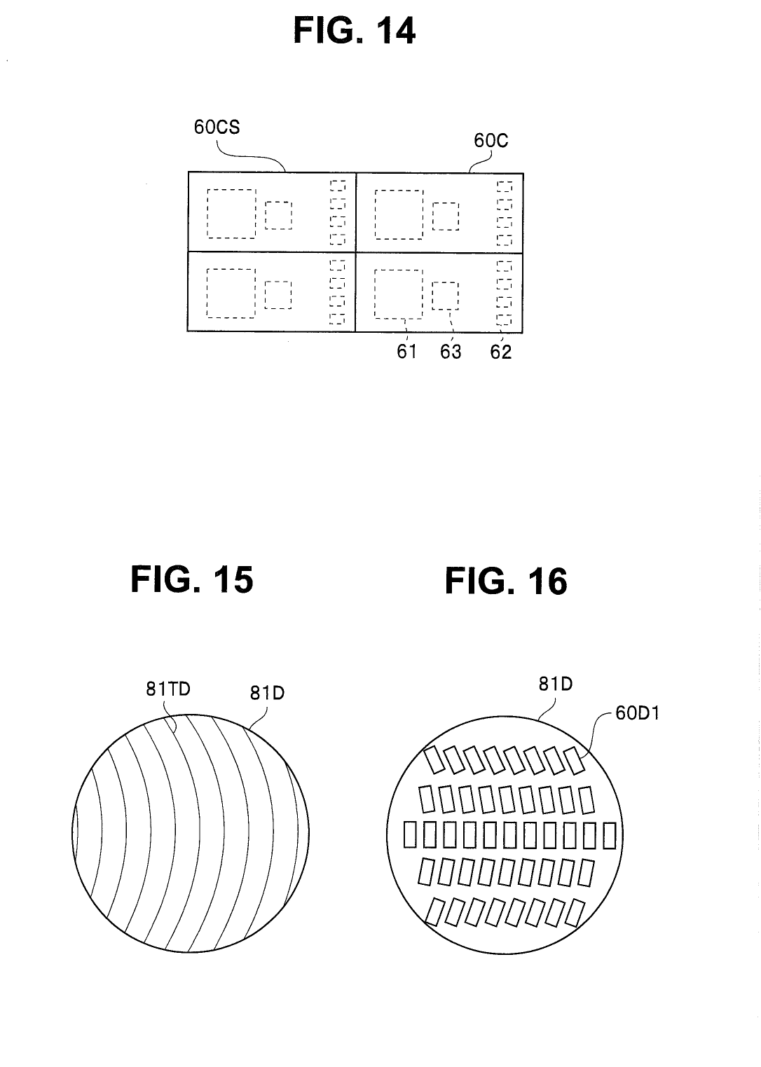

Note that in the example described above, the grinding is performed after the silicon wafer is divided into the individual image pickup substrates 60C1. However, as shown in FIG. 14, the silicon wafer may be cut into workpieces 60CS including the plurality of image pickup substrates 60C1. The workpieces 60CS may be ground and then divided into the individual image pickup substrates 60C.

The workpieces 60CS are disposed on the holding plate 81 such that the directions of the plurality of grooves 60TC (saw marks) formed on all of the image pickup substrates 60C1 included in the workpieces 60CS are a predetermined direction.

### <Modification of Second Embodiment>

A creep feed type processing machine may be used as a grinding machine to thin the image pickup substrates 60C1.

Saw marks 80TD shown in FIG. 15 are formed on a holding plate 81D in the creep feed type processing machine.

Therefore, as shown in FIG. 16, a plurality of image pickup substrates 60D1 of an image pickup apparatus 10D of modification 1 are disposed on the holding plate 81D such that the directions of the saw marks to be formed are a predetermined same direction.

In the image pickup apparatus 10, the directions of the grooves of the image pickup substrate 60D1 are inclined more than 45 degrees relative to the short axis direction. Therefore, a crack or the like does not occur on the side surface in the major axis direction, and the manufacturing yield is high.

That is, in the manufacturing method of the image pickup apparatus for endoscope of the embodiment, the grinding method is preferably centerless infeed grinding from the viewpoint of productivity. However, the grinding method is not limited to the infeed grinding, and a creep feed type processing machine or the like may be used as long as processing is possible so that the directions of the saw marks are inclined more than 45 degrees relative to the short axis direction.

Furthermore, the grinding may be performed without considering the directions of the saw marks to be formed, and grinding may be further applied only to the region facing the region to which the image pickup optical system 20 (prism 30) is not adhered to form the saw marks inclined more than 45 degrees relative to the short axis direction.

Although the endoscope is for medical use in the description of the embodiments, the endoscope is not limited to an endoscope for medical use, and it is obvious that the embodiments can be applied to an endoscope for industrial use with a small diameter.

Furthermore, although the image pickup substrate 60 is rectangular in plan view in the description, the shape is not limited to an accurate rectangle, and for example, four corners may be chamfered.

The present invention is not limited to the embodiments and the like described above, and various changes, modifications, and the like can be made without changing the scope of the present invention.

What is claimed is:

1. An image pickup apparatus for endoscope comprising:
an image pickup optical system;
an optical path conversion element configured to receive light from the image pickup optical system and bend an optical path; and
an image pickup substrate rectangular in plan view with a thickness equal to or greater than 20 μm and equal to or smaller than 100 μm, in which the optical path conversion element is adhered to a first main surface, and a light receiving portion configured to receive the light bent by the optical path conversion element is formed, wherein
at least one groove is formed on a second main surface of the image pickup substrate, and a direction of the groove is inclined more than 45 degrees relative to a short axis direction of the image pickup substrate.

2. The image pickup apparatus for endoscope according to claim 1, wherein
the groove is orthogonal to the short axis direction.

3. The image pickup apparatus for endoscope according to claim 2, wherein
a depth of the groove is equal to or greater than 10% and equal to or smaller than 50% of the thickness of the image pickup substrate.

4. The image pickup apparatus for endoscope according to claim 2, wherein
the groove is formed in a region other than a region equivalent to a back surface of the light receiving portion in the second main surface.

5. The image pickup apparatus for endoscope according to claim 1, wherein
the groove is a saw mark formed during grinding of the second main surface of the image pickup substrate.

6. The image pickup apparatus for endoscope according to claim 5, wherein
a surface roughness Rz of the second main surface in a direction orthogonal to the groove is equal to or greater than 1 μm and equal to or smaller than 5 μm.

7. The image pickup apparatus for endoscope according to claim 1, wherein
a direction of the groove in a region of the second main surface facing a region other than a region where the optical path conversion element is adhered is a direction different from a groove of a saw mark formed during grinding for thinning.

8. A manufacturing method of an image pickup apparatus, the image pickup apparatus comprising:
an image pickup optical system;
an optical path conversion element configured to receive light from the image pickup optical system and bend an optical path; and
an image pickup substrate rectangular in plan view with a thickness equal to or greater than 20 μm and equal to or smaller than 100 μm, in which the optical path conversion element is adhered to a first main surface, and a light receiving portion configured to receive the light bent by the optical path conversion element is formed, the manufacturing method comprising steps of:
forming a plurality of light receiving portions on a first main surface of a semiconductor substrate;
manufacturing a plurality of image pickup substrates by cutting the semiconductor substrate;
disposing the plurality of image pickup substrates on a grinding machine such that directions of saw marks to be formed are same; and
forming grooves inclined more than 45 degrees relative to a short axis direction of the plurality of image pickup substrates by grinding second main surfaces of the plurality of image pickup substrates.

9. The manufacturing method of the image pickup apparatus for endoscope according to claim 8, wherein
the grinding is centerless infeed grinding.
